(19) United States
(12) Patent Application Publication
(10) Pub. No.: US 2013/0281952 A1
(43) Pub. Date: Oct. 24, 2013

(54) WOUND DRESSING SYSTEM

(71) Applicant: SILITECH TECHNOLOGY CORPORATION, NEW TAIPEI CITY (TW)

(72) Inventors: CHI SHENG CHU, NEW TAIPEI CITY (TW); CHIN-CHIEN HSIEH, NEW TAIPEI CITY (TW)

(21) Appl. No.: 13/919,138

(22) Filed: Jun. 17, 2013

Related U.S. Application Data
(63) Continuation-in-part of application No. 13/354,648, filed on Jan. 20, 2012.

(51) Int. Cl. A61L 15/60

(52) U.S. Cl. CPC A61L 15/60
USPC 604/372

(57) ABSTRACT

A wound dressing system which includes an injection device, a plurality of substantially spherical hydrogel particles having a diameter ranging between 1 to 4 mm, and a cover layer being adherent to skin. The hydrogel particles are made of acrylate polymer containing sulfonate. The plurality of hydrogel particles are injected to a cavity by the injection device and the cavity is sealed by the cover layer.

This application is a continuation-in-part of U.S. patent application Ser. No. 13/354,648, filed Jan. 20, 2012. The entire disclosure is incorporated by reference for all purposes as if fully set forth herein.

### BACKGROUND

1. Field of the Invention

The instant disclosure relates to a wound dressing system; in particular, to a wound dressing system utilizing an injection device to load granules onto a wound.

2. Description of Related Art

To promote healing process of an external wound, different types of dressings are used. The commonly used forms of current wound dressings are powder, foam, porosity, non-woven dressings and the like.

Powder dressings are normally used on wounds of large surface area. However, when the wound decorated by powder is in contact with fluid, clot can easily occur because the powder is attached to the newly developed tissue. As a result, the powder is difficult to remove without irritating the patient.

In the case of foam dressings, a wound is sealed under the layer to prevent foreign particle invasion and maintain certain degree of moisture within the wound. However, foam dressing is configured to a plane and the protection is limited to the surface of the wound. When a wound has a depth, i.e., wound with a cavity, the foam dressing cannot reach the heart of the wound.

The porosity dressings are capable of absorbing wound exudates so as to prevent the wounds from inflaming and maintain moisture within the wound. However, after absorbing great amount of exudates, the porosity dressings are difficult to be removed because the surface of the porosity dressings is prone to attach to the epidermal layer.

Non-woven dressings include bio-macromolecular materials which facilitate wound healing by releasing associated chemicals. Specifically, bio-macromolecules are digested by enzymes (for example, collagen to collagenase, glycoside to lysozyme). The broken down molecules can enhance fibroblast proliferation, and fibroblast is critical in wound healing. However, the structure of the non-woven dressings are often deformed upon absorbing wound exudates, resulting in adhesion to the wound bed. Hence, removing the non-woven dressings may incur secondary damage to the wound.

Furthermore, please refer to FIG. 1. A wound W with a cavity is formed. Conventional dressings 12 are applied directly on the wound W over the skin P. The dressings 12 cover the surface of the wound whereas the entire wound (i.e., the cavity portion) is not fully attended. Furthermore, the dressings 12 cannot be easily removed from the wound W because the adhesion to the skin P is relatively aggressive. Also, the dressings 12 fail to maintain appropriate moisture balance of the wound W. Specifically, the wound is concealed from the atmosphere while the wound exudates are not properly drained.

Pawelchack et al. disclosed dressings, granules, and their use in treating wounds in U.S. Pat. No. 4,538,603. The granules are dried at 55° C. for 24 hours in the manufacturing process and limited to 10 to 40 mesh particle size. The drying processing and the powder-like particle size increase the complexity in manufacturing. In addition, the granules form a gel-like mass after hydrated. The small particle size may be easily trapped or stuck in the wound and difficult to be thoroughly removed without more aggressive procedure.

### SUMMARY OF THE INVENTION

The instant disclosure provides a wound dressing system which includes an injection device, a plurality of substantially spherical hydrogel particles having a diameter ranging between 1 to 4 mm, and a cover layer being adherent to skin. The hydrogel particles are made of acrylate polymer containing sulfonate. The plurality of hydrogel particles are loaded to a cavity by the injection device and the cavity is sealed by the cover layer.

The hydrogel particles exhibits great flexibility and therefore fill the cavity in conformity with its shape. Also, water is retained in the particle core and exudate is absorbed by the hydrogel particles as well. The hydrogel particles maintain preferable moisture in terms of wound healing.

In order to further understand the instant disclosure, the following embodiments are provided along with illustrations to facilitate the appreciation of the instant disclosure; however, the appended drawings are merely provided for reference and illustration, without any intention to be used for limiting the scope of the instant disclosure.

### BRIEF DESCRIPTION OF THE DRAWINGS

FIG. 1 shows a schematic diagram of a conventional wound dressings;

FIG. 2 shows a schematic view of a plurality of hydrogel particles of a wound dressings system filling up the wound in accordance with an embodiment of the instant disclosure;

FIG. 3 shows a schematic view of a dressings covering the wound in accordance with an embodiment of the instant disclosure;

FIG. 4 shows a schematic view of a plurality of hydrogel particles of a wound dressings system swelling upon absorbing the wound exudates in accordance with an embodiment of the instant disclosure;

FIG. 5 shows an enlarged view of FIG. 4;

FIG. 6 shows a schematic view of removing a plurality of hydrogel particles in accordance with an embodiment of the instant disclosure; and

FIG. 7 shows three electro microscopic diagrams of tissue slices over wound healing after being treated by a plurality of hydrogel particles. The tissues under or after healing are intact and no particles remain in the tissue.

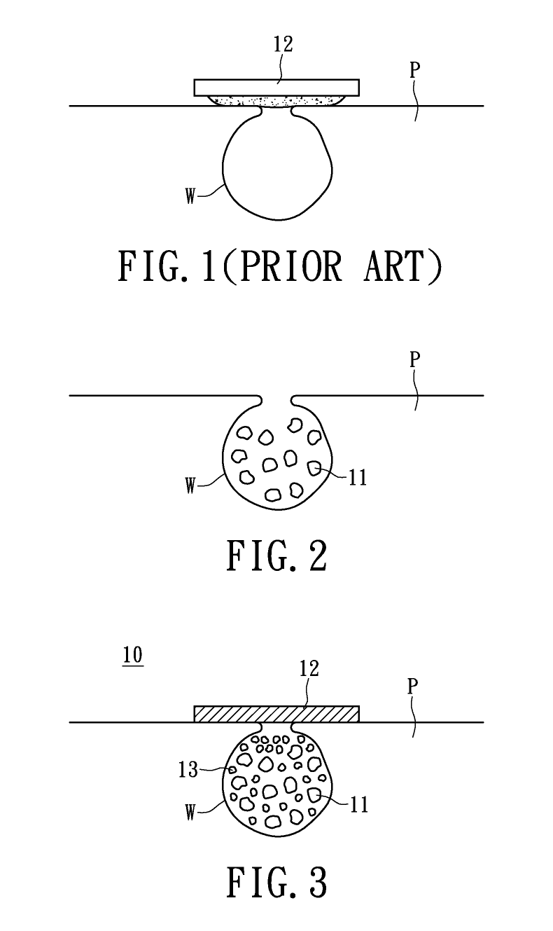

### DETAILED DESCRIPTION OF THE PREFERRED EMBODIMENTS

The aforementioned illustrations and following detailed descriptions are exemplary for the purpose of further explaining the scope of the instant disclosure. Other objectives and advantages related to the instant disclosure will be illustrated in the subsequent descriptions and appended drawings.

The instant disclosure provides a wound dressings system 10. The wound dressings system 10 includes a plurality of hydrogel particles 11, an injection device and a cover layer 12.

The hydrogel particle is substantially spherical and has a diameter ranging between 1 to 4 mm, preferably 2 to 3 mm. The core structure of the hydrogel is an acrylate polymer containing hydroxyl group and sulfonate. Hydrogel is a highly hydrophilic polymer having polar functional groups. After ultra violet (UV) light cross linking, the polymers form a net or a cage structure. In particular, the cage structure is flexible and bendable. Under normal conditions the intermolecular force allows the polymer being slightly bent. When heat, liquid or pH value changes, the intermolecular force may be interrupted and the polymer expands accordingly. The hydrophilic and polar main chain can bond water through hydrogen bond. The cage structure can further retain free water molecules therein. Among the hydrophilic functional groups of the polymer, sulfonate exhibits strong polarity and therefore attracts water molecules effectively. It is worth noting that the hydrogel particles 11 do not go through drying process in fabrication. Specifically, the hydrogel contains more than 80% by weight of water before processing. The spherical configuration is obtained due to the surface tension of the hydrogel, namely intermolecular cohesion. This process is similar to water drop formation. Consequently, a great portion of water is therefore preserved in the hydrogel particles. In other words, by omitting the step of drying in fabrication, the water retained by the hydrogel does not evaporate to the air in manufacturing and the surface of the hydrogel particles 11 are moist.

Furthermore, the hydrogel particles 11 are deformable and may expand in volume. Specifically, the wound dimension or configuration does not prevent the hydrogel particles 11 from filling the cavity thoroughly because the hydrogel particles 11 are highly flexible. That is to say, the shape of the hydrogel particles 11 changes according to the cavity structure. In addition, after absorbing aqueous solution, the hydrogel particles may swell 20 times larger than its initial size. More specifically, the initial diameter may increase to 3 times longer. As a result, the volume increase to 27 times larger because of the cube of 3. For example, the diameter of a particle may increase from 1 mm to 3 mm after absorbing exudate. The volume of one hydrogen particle 11 then increases to 27 mm³. The resulting diameter may increase to 3 to 11 mm, and preferably falls between 5 to 8 mm. The swelling dimension of the hydrogel particles 11 is controlled in this particular range because the wound has to be filled sufficiently yet the swelled hydrogel particles 11 should not apply excessive pressure to the wound.

A wound W goes through difference phases in the healing process. In the stage of tissue regeneration, large amount of exudate is excreted to the wound bed. The excessive amount of exudate may result in further pathogen infection, oedema and developing of ulcer, and therefore removing excessive exudate from the wound can maintain a reasonably damp but not wet healing environment. The hydrogel particles 11 are injected to the wound W by the injection device. The hydrogel particles 11 can smoothly go through the narrow needle or injection tip of a syringe because the deformation is temporary and the particles resume their spherical shape once released from the tube. By syringe injection, the hydrogel particles 11 can have access to a deep wound having a small skin opening. The loading volume of hydrogel particles 11 can be precisely controlled according to the scale shown on the syringe tube. However, the hydrogel particles 11 may be deposited to the cavity by pouring with or without a funnel or a tube, tweezers or the like. A wound care agent 13 can then be added and mixed with the hydrogel particles 11. The wound care agent 13 may be anti-inflammatory agent, pain relieving agent, growth hormone, antibiotics, anti-infective agent, or other drugs which promote healing. After the hydrogel particles 11 fill the cavity and the wound care agent 13 is added, the cover layer 12 seals the wound opening, as shown in FIG. 3. The cover layer 12 can be made of natural fiber, artificial fiber, woven or non-woven fabric or the like. The cover layer 12 may be attached to the skin by bandage or tape. Alternatively, the cover layer 12 may be adherent to the skin and is capable of attaching to the skin upon contacting.

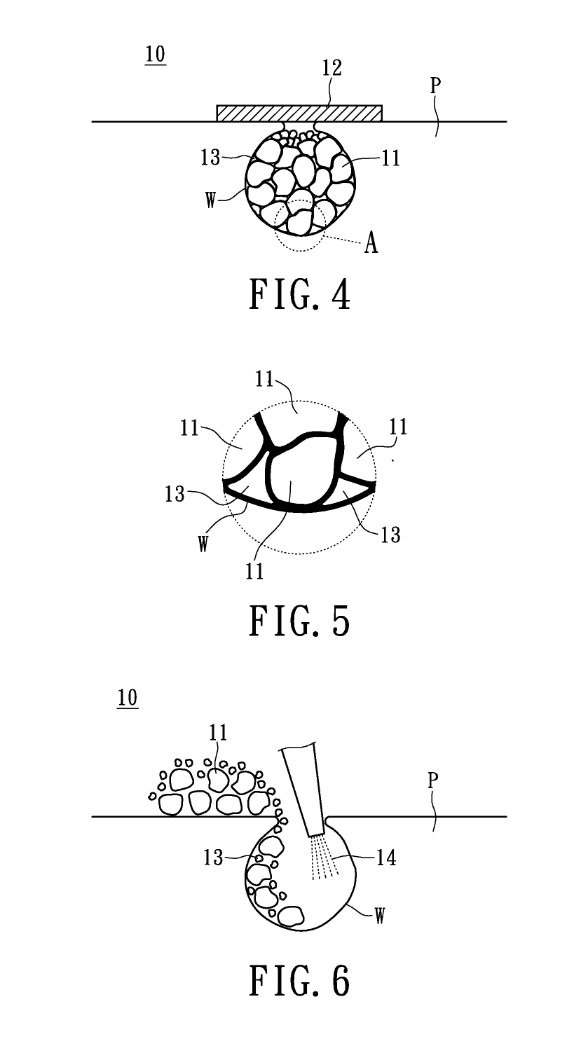

In the healing period, the hydrogel particles 11 slightly deform in conformity with the topology of the wound bed such that the cavity is completely attended over its surface. In addition, the hydrogel particles 11 exhibit desirable adherence which comes from the intermolecular interaction to the tissue. In other words, once the hydrogel particles 11 are injected to the cavity, the particles are loosely attached to the wound bed. Nevertheless, the cover layer 12 ensures the hydrogel particles 11 remain in the cavity and provides an additional barrier for protecting the wound from contamination. Also, the cover layer 12 may absorb exudate generated over the surface region of the wound W. It should be noted that the cover layer 12 does not interact with the hydrogel particles 11. In other words, the hydrogel particles 11 are not attached to the cover layer 12. The cover layer 12 serves as a lid over the opening of the wound W. In FIG. 4, the hydrogel particles 11 swell because excessive exudate is absorbed by the strong absorbent acrylate polymer core of the hydrogel particles 11. The hydrogel particles 11 become saturated and expand in volume to retain the fluid. It is worth mentioning that the surface of the hydrogel particles 11 remains moist throughout the healing period. The acrylate polymer core provides effective fluid retention for both exudate and water. Specifically, the hydrogel particles 11 intake body fluid while the water molecules locked by the hydrophilic functional groups still exist. The healing environment is therefore kept moist and it has been widely acknowledged that a damp instead of wet condition is favorable in wound healing. Furthermore, the wound care agent 13 promotes related wound healing chemical reaction and the overall healing period can be shortened as well.

Attention is now invited to FIG. 5, showing an enlarged view of region A of FIG. 4. The swollen hydrogel particles 11 make even tighter contact with the wound bed. Because of the flexibility of hydrogel particles 11, they can closely line the irregular wound bed leaving no gaps behind. In wound healing, the debris is firstly removed, and the factors that cause cell migration, proliferation and differentiation are attracted to the site. The emergence of fibroblasts marks the onset of cell regeneration. Fibroblasts migrate within the wound site and proliferate as well. The presence of the hydrogel particles 11 facilitates fibroblast migration because of its moist surface and provides attachment site to the fibroblasts. Fibroblasts make the essential proteins found in extracellular matrix which is the structural support to tissues. Once the fibroblasts anchor, associated growth factors are also drawn to the wound site. The wound healing related cells and chemicals then carry on to granulation tissue formation. In general, hydrogel particles 11 provide a moist healing environment that facilitates fibroblasts in migration as well as proliferation, and therefore other associated molecules aggregate to the wound site in a shorter time.

As mentioned before, the hydrogel particles 11 show great advantage of fluid retention. When the wound W is dry, water molecules locked by the hydrogel particles 11 are released to the wound site by diffusion. That is to say, water goes along the concentration gradient and is freed from the hydrogel particles 11. The wound W can then be kept moist which favors cell activation and proliferation. Additionally, the hydrogel particles 11 can also be used as fillers. For example, the hydrogel particles 11 can be injected to any cavity in the body and fill up the space. In short, the hydrogel particles 11 are capable of maintaining appropriate moist environment.

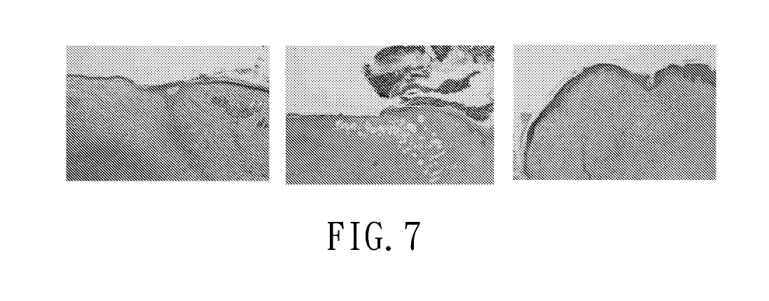

Upon removal of the hydrogel particles 11, a removal agent 14 is used. The removal agent 14 can be normal saline, distilled water, pure water, or the like. The hydrogel particles 11 can be easily washed away from the cavity as shown in FIG. 6. Unlike the conventional wound dressing, the hydrogel particles 11 does not show aggressive adhesion to the wound bed and therefore can be rinsed off by liquid. Specifically, the cage structure of the acrylate polymer shows great integrity even after absorbing exudate such that the hydrogel particles 11 do not cross link with the newly generated tissue or stick to the wound bed. The removal of hydrogel particles 11 is fast and pain-free because the particles can be completely cleaned up by liquid washing. FIG. 7 shows three the tissue slices at different wound healing phases. The wound was treated by the hydrogel particles 11 and the wound bed remained intact and showed no sign of particle remainders.

Additionally, the hydrogel particles 11 may act as a functional factor carrier. Any functional factors which are water soluble can be mixed in the raw hydrogel material given the factor is dissolved first.

In summary, the wound dressings system of the instant disclosure using injection device to load the hydrogel particles. The hydrogel particles loosely attach to the wound bed and swell when absorbing exudates. The hydrogel particles are also capable of releasing water to the wound site and maintain an appropriate balance of moisture. During cell regeneration, the hydrogel particles serve as an attachment core or migration intermediate. The related cells and molecules are then attracted to the wound site and wound healing is thus accelerated. Moreover, the smooth surface of the hydrogel particles and their integrity ensure an easy removal by liquid washing. The hydrogel particles and the wound dressings system have been proved clinically effective in wound treatment.

The descriptions illustrated supra set forth simply the preferred embodiments of the instant disclosure; however, the characteristics of the instant disclosure are by no means restricted thereto. All changes, alternations, or modifications conveniently considered by those skilled in the art are deemed to be encompassed within the scope of the instant disclosure delineated by the following claims.

What is claimed is:

1. A wound dressings system comprising:
an injection device;
a plurality of substantially spherical hydrogel particles having a diameter ranging between 1 to 4 mm and made of acrylate polymer containing sulfonate; and
a cover layer adherable to skin;
wherein the plurality of hydrogel particles are injected into a wound cavity through the injection device, and the cavity is sealed by the cover layer.

2. The wound dressings system according to claim 1, wherein the diameter of the hydrogel particles increases no more than 11 mm after absorbing liquid.

3. The wound dressings system according to claim 1, wherein the hydrogel particles contains approximately 80% by weight of water.

4. The wound dressings system according to claim 1, wherein the acylate polymer is configured to a cage structure by ultra violet light cross linking.

5. The wound dressings system according to claim 4, wherein the acrylate polymer is flexible to allow the hydrogel particles to form to a shape in conformity with the cavity.

6. The wound dressings system according to claim 1, wherein the hydrogel particles has an effect of releasing water.

7. The wound dressings system according to claim 1, wherein the injection delivery device is a syringe.

8. The wound dressings system according to claim 1, wherein the hydrogel particles carry functional factors.
